(19) United States
(12) Patent Application Publication
(10) Pub. No.: US 2026/0263141 A1
(43) Pub. Date: Sep. 10, 2026

(54) MULTI-FUNCTION ELECTROSURGICAL DEVICE, AND ASSOCIATED SYSTEMS AND METHODS

(71) Applicant: Boston Scientific Scimed, Inc., Maple Grove, MN (US)

(72) Inventors: Austin Grant Johnson, Worcester, MA (US); Prashanth T. Somasundaram, Chicago, IL (US)

(73) Assignee: Boston Scientific Scimed, Inc., Maple Grove, MN (US)

(21) Appl. No.: 19/558,984

(22) Filed: Mar. 6, 2026

Related U.S. Application Data
(60) Provisional application No. 63/768,386, filed on Mar. 7, 2025.

(51) Int. Cl. A61B 18/12; A61B 18/00; A61B 18/14

(52) U.S. Cl. CPC A61B 18/1206; A61B 18/1445; A61B 18/1492; A61B 2018/00077; A61B 2018/00083; A61B 2018/00178; A61B 2018/00184; A61B 2018/00589; A61B 2018/00601; A61B 2018/00607; A61B 2018/00702; A61B 2018/126; A61B 2218/002

(57) ABSTRACT

A bipolar electrosurgical device having first and second electrodes which are movable with respect to each other to be positionable in different configuration for operating in different modes. In one configuration, the bipolar electrosurgical device may operate as an electrosurgical cutting device. In another configuration, the bipolar electrosurgical device may operate as an electrocoagulation device. The bipolar electrosurgical device may also operate in further modes without the application of energy thereto. The first electrode may be in the form of a cutting device of an electrosurgical cutting device, and the second electrode may be pivotable with respect to the first electrode.

### CROSS-REFERENCE TO RELATED APPLICATIONS

This application claims the benefit of priority under 35 U.S.C. § 119 to U.S. Provisional Application No. 63/768,386, filed Mar. 7, 2025, the entire disclosure of which is hereby incorporated by reference herein for all purposes.

### FIELD

The present disclosure relates generally to the field of medical devices, systems, and methods used in applying energy to a patient such as for therapeutic purposes. More particularly, the present disclosure relates to bipolar electrosurgical devices, systems, and methods configured to perform in more than one mode of operation during a medical procedure.

### BACKGROUND

During endoluminal surgical (ELS) procedures, physicians and other medical professionals use a host of tools to perform operations with respect to anatomical tissue. One of the tools utilized is an electrocautery cutting knife. Electrocautery cutting knives can cut through tissue efficiently and precisely, and may be capable of being held open at length or locked into position. Some electrocautery cutting knives are also equipped with the capability to inject water, a lifting agent, or other solution, such as to facilitate cutting of the interface between tissue layers (e.g., between muscularis and submucosal tissue layers). Endoscopists frequently use monopolar cutting instruments to perform endo-surgical cuts on tissue. When used with endoscopic submucosal dissection (ESD) or endoscopic mucosal resection (EMR), these electrosurgical knives often have a flushing lumen that acts to perform submucosal lifting of tissue to form a “bleb”. Bipolar cutting instruments typically cause less tissue damage compared to monopolar cutting instruments, which are more likely to cause thermal damage to surrounding tissue because of their inherent mechanism of action, requiring a grounding pad at a distance from the active electrode, and thus passage of current through the patient's body and not in the region of the treatment site.

During endoscopic procedures, if there is any sort of bleeding, such as caused by the cutting instrument, medical professionals often use coagulation graspers to coagulate bleeding blood vessels. In some instances, medical professionals coagulate blood vessels prior to performing a cut as a precautionary measure. In the context of ESD, because of the tendency to cut blood vessels, physicians frequently have to swap or exchange a cutting device for coagulation-graspers when cutting through tissue. This is a time-consuming and frustrating process and solutions to these and other challenges in the art would be welcome. It is with respect to these and other considerations that the present improvements may be useful.

### SUMMARY

This Summary is provided to introduce, in simplified form, a selection of concepts described in further detail below in the Detailed Description. This Summary is not intended to necessarily identify key features or essential features of the claimed subject matter, nor is it intended as an aid in determining the scope of the claimed subject matter. Those of ordinary skill in the art will understand that each of the various aspects and features of the present disclosure may advantageously be used separately in some instances, or in combination with other aspects and features of the disclosure in other instances, whether or not described in this Summary. No limitation as to the scope of the claimed subject matter is intended by either the inclusion or non-inclusion of elements, components, or the like in this Summary.

In some aspects, a bipolar electrosurgical device formed in accordance with various principles of the present disclosure includes a first electrically-conductive component and a second electrically-conductive component, and the first electrically-conductive component and the second electrically-conductive component are movable with respect to each other between at least a first configuration and a second configuration.

In some aspects, the second electrically-conductive component is pivotable with respect to the first electrically-conductive component.

In some aspects, the level of energy supplied to the bipolar electrosurgical device is selectable by a medical professional based on the configuration of the electrically-conductive components.

In some aspects, in the first configuration of the electrically-conductive components, the electrically-conductive components are usable as an electrosurgical cutting device. In some aspects, in the second configuration of the electrically-conductive components, the electrically-conductive components are usable as an electrocoagulation device with respect to tissue positioned between the first and second electrically-conductive components.

In some aspects, in the second configuration of the electrically-conductive components, the electrically-conductive components are usable as an electrocoagulation device with respect to tissue positioned between the first and second electrically-conductive components. In some aspects, the second electrically-conductive component has a surface configured to facilitate grasping of tissue between the second electrically-conductive component and the first electrically-conductive component.

In some aspects, the first electrically-conductive component is configured as a cutting device. In some aspects, the first electrically-conductive component defines a lumen therethrough for delivery of a fluid therethrough and out the distal end of the bipolar electrosurgical device.

In some aspects, the bipolar electrosurgical device includes an elongate control element operably coupled with a proximal end of the second electrically-conductive component and extendable to a proximal end of the bipolar electrosurgical device for application of a force thereto to move the second electrically-conductive component with respect to the first electrically-conductive component.

In some aspects, the bipolar electrosurgical device includes an insulative component between a portion of the first electrically-conductive component and a portion of the second electrically-conductive component. In some aspects, the first electrically-conductive component is electrically coupled with an electrically conductive hub, and the insulative component is overmolded over the electrically conductive hub and over a portion of a pivot pin operably coupled with a proximal end of the second electrically-conductive component to pivotably couple the second electrically-conductive component with respect to the first electrically-conductive component.

In some aspects,, a bipolar electrosurgical cutting device formed in accordance with various principles of the present disclosure includes a first electrode configured to cut tissue and a second electrode, and the first electrode and the second electrode are movable with respect to each other.

In some aspects, the second electrode is pivotable with respect to the first electrode.

In some aspects, the first electrode and the second electrode are movable to a first configuration in which the first electrode and the second electrode are operable to cut tissue with energy supplied to the first electrode and passing through tissue, contacted by the first electrode and the second electrode, to the second electrode.

In some aspects, the first electrode and the second electrode are movable to a second configuration in which the first electrode and the second electrode are operable to coagulate tissue positioned therebetween with energy supplied to the first electrode and passing through the tissue and to the second electrode.

In some aspects, a method of performing a procedure with respect to tissue within a patient includes, in accordance with various principles of the present disclosure, delivering an operable element of an electrosurgical device transluminally into a patient and to a treatment site, the operable element comprising a first electrode and a second electrode movable with respect to each other; providing energy to the first electrode and the second electrode when the operable element is in a first configuration to operate in a first mode with respect to tissue at the treatment site; and providing energy to the first electrode and the second electrode when the operable element is in a second configuration to operate in a second mode with respect to tissue at the treatment site.

In some aspects, the method further includes providing a first level of energy to the operable element when in the first configuration, and a second level of energy, different from the first level of energy, to the operable element when in the second configuration.

In some aspects, the method further includes cutting tissue in the first mode of operation.

In some aspects, the method further includes coagulating tissue in the second mode of operation.

These and other features and advantages of the present disclosure, will be readily apparent from the following detailed description, the scope of the claimed invention being set out in the appended claims. While the following disclosure is presented in terms of aspects or embodiments, it should be appreciated that individual aspects can be claimed separately or in combination with aspects and features of that embodiment or any other embodiment.

### BRIEF DESCRIPTION OF THE DRAWINGS

Non-limiting embodiments of the present disclosure are described by way of example with reference to the accompanying drawings, which are schematic and not intended to be drawn to scale. The accompanying drawings are provided for purposes of illustration only, and the dimensions, positions, order, and relative sizes reflected in the figures in the drawings may vary. For example, devices may be enlarged so that detail is discernable, but is intended to be scaled down in relation to, e.g., fit within a working channel of a delivery catheter or endoscope. For purposes of clarity and simplicity, not every element is labeled in every figure, nor is every element of each embodiment shown where illustration is not necessary to allow those of ordinary skill in the art to understand the disclosure.

The detailed description will be better understood in conjunction with the accompanying drawings, wherein like reference characters represent like elements, as follows:

FIG. 1 illustrates a perspective view of an example of an embodiment of an electrosurgical device formed in accordance with aspects of the present disclosure.

FIG. 2 illustrates a detail perspective view along detail II of FIG. 1, illustrating an example of an embodiment of an operable element of an electrosurgical device formed in accordance with various aspects of the present disclosure and in a first configuration.

FIG. 3 illustrates a view similar to that of FIG. 2 but in a second configuration.

FIG. 4 illustrates a cross-sectional view along line IV-IV of FIG. 3.

FIG. 5 illustrates a view similar to that of FIG. 3 but from a different perspective.

### DETAILED DESCRIPTION

The following detailed description should be read with reference to the drawings, which depict illustrative embodiments. It is to be understood that the disclosure is not limited to the particular embodiments described, as such may vary. All apparatuses and systems and methods discussed herein are examples of apparatuses and/or systems and/or methods implemented in accordance with one or more principles of this disclosure. Each example of an embodiment is provided by way of explanation and is not the only way to implement these principles but are merely examples. Thus, references to elements or structures or features in the drawings must be appreciated as references to examples of embodiments of the disclosure, and should not be understood as limiting the disclosure to the specific elements, structures, or features illustrated. Other examples of manners of implementing the disclosed principles will occur to a person of ordinary skill in the art upon reading this disclosure. In fact, it will be apparent to those skilled in the art that various modifications and variations can be made in the present disclosure without departing from the scope or spirit of the present subject matter. For instance, features illustrated or described as part of one embodiment can be used with another embodiment to yield a still further embodiment. Thus, it is intended that the present subject matter covers such modifications and variations as come within the scope of the appended claims and their equivalents.

It will be appreciated that the present disclosure is set forth in various levels of detail in this application. In certain instances, details that are not necessary for one of ordinary skill in the art to understand the disclosure, or that render other details difficult to perceive may have been omitted. The terminology used herein is for the purpose of describing particular embodiments only, and is not intended to be limiting beyond the scope of the appended claims. Unless defined otherwise, technical terms used herein are to be understood as commonly understood by one of ordinary skill in the art to which the disclosure belongs. All of the devices and/or methods disclosed and claimed herein can be made and executed without undue experimentation in light of the present disclosure.

As used herein, “proximal” refers to the direction or location closest to the user (medical professional or clinician or technician or operator or physician, etc., such terms being used interchangeably herein without intent to limit, and including automated controller systems or otherwise), etc., such as when using a device (e.g., introducing the device into a patient, or during implantation, positioning, or delivery), and/or closest to a delivery device, and “distal” refers to the direction or location furthest from the user, such as when using the device (e.g., introducing the device into a patient, or during implantation, positioning, or delivery), and/or closest to a delivery device. “Longitudinal” means extending along the longer or larger dimension of an element. A “longitudinal axis” extends along the longitudinal extent of an element, though is not necessarily straight and does not necessarily maintain a fixed configuration if the element flexes or bends, and “axial” generally refers to along the longitudinal axis. However, it will be appreciated that reference to axial or longitudinal movement with respect to the above-described systems or elements thereof need not be strictly limited to axial and/or longitudinal movements along a longitudinal axis or central axis of the referenced elements. “Central” means at least generally bisecting a center point and/or generally equidistant from a periphery or boundary, and a “central axis” means, with respect to an opening, a line that at least generally bisects a center point of the opening, extending longitudinally along the length of the opening when the opening comprises, for example, a tubular element, a channel, a cavity, or a bore. As used herein, a “lumen” or “channel” or “bore” or “passage” is not limited to a circular cross-section. As used herein, a “free end” of an element is a terminal end at which such element does not extend beyond. It will be appreciated that terms such as at or on or adjacent or along an end may be used interchangeably herein without intent to limit unless otherwise stated, and are intended to indicate a general relative spatial relation rather than a precisely limited location. Finally, reference to “at” a location or site is intended to include at and/or about the vicinity of (e.g., along, adjacent, proximate, etc.) such location or site. As understood herein, corresponding is intended to convey a relationship between components, parts, elements, etc., configured to interact with or to have another intended relationship with one another.

Various devices, assemblies, systems, and methods exist for energy-based medical treatment and/or performance of one or more medical/surgical procedures. For instance, electrosurgical cutting devices may utilize electrical energy (e.g., high frequency electrical current) to cut tissue. The energy which is passed through the electrosurgical knife meets resistance when the electrosurgical knife is contacted with (or brough sufficiently close to) tissue, and may be converted into heat (e.g., at the point of contact) which may be used to cut the tissue (e.g., by vaporizing the water in the tissue cells). In some aspects, the high heat of the electrosurgical knife may coagulate surrounding tissue to stop any bleeding resulting from cutting the tissue. The amount of energy transmitted to the device may be modified to selectively primarily cut tissue (e.g., application of high frequency current) or to selectively primarily coagulate tissue (e.g., pulsed or intermittent or lower frequency current). Some electrosurgical devices are dedicated simply to coagulation. An electrocoagulation device typically passes high-frequency electrical current through tissue to cause resistive heating within the tissue to result in coagulation of the tissue, such as to seal of blood vessels and/or to promote clotting and/or to control bleeding. Electrocauterization devices utilize a heated electrode which is directly contacted with tissue to cauterize (seal or destroy) the tissue, generally without passing electrical current through the tissue.

There has been a growing interest in the medical field in minimally-invasive procedures, e.g., transluminal, transcatheter, endoscopic, etc., which do not require open surgery (cutting open the patient), but, instead, access a target site within the patient via a natural orifice (or, in some instances, a small incision not considered to constitute an open-surgery cut). Thus, one trend in medical field includes moving from laparoscopic and open surgical procedures to miniaturized, minimally-invasive procedures such as endoscopic procedures. Medical instruments designed for use in such procedures must be small enough and flexible enough to be inserted into a natural opening or orifice (or small incision) in the patient and navigated within the patient (e.g., through curved and/or tortuous passages) to a target site. It will be appreciated that terms such as medical instruments, tools, devices, accessories, etc., may be used interchangeably herein without intent to limit. It will further be appreciated that reference may be made to a target site, treatment site, anatomical site, delivery site, deployment site, implant/implantation site, site of implantation, etc., interchangeably and without intent to limit. Typically, because of the limited amount of space within the patient's body to perform such procedures (without cutting open the patient), various size constraints are imposed. Most endoscopes have a single working channel, which may not accommodate more than just one or two medical instruments at a time. Various device exchanges, with a device selected for performing one aspect of a procedure being used, and then withdrawn, so that another device selected for performing another aspect of the procedure may be inserted into the patient and utilized until yet another device is needed.

In accordance with various principles of the present disclosure, an electrosurgical device as described herein has an operable end configured to perform more than one different type of action during a medical procedure. As such, the electrosurgical device reduces the number of device exchanges which otherwise would be necessary during a procedure.

Electrosurgical devices may be monopolar or bipolar. Currently, most electrosurgical cutting devices are monopolar. Monopolar devices utilize a single active electrode which is electrically connected to an electrosurgical energy generator which delivers current to the active electrode. The active electrode is contacted with tissue at a treatment site to deliver electrical current to the tissue to perform the function of the device (e.g., to cut or coagulate tissue). The current passes through the active electrode and through the tissue and is returned to a grounding pad positioned outside the patient (e.g., on the patient's skin) to complete the electrical circuit. The grounding pad may help disperse current and prevent burns or injury by ensuring the current does not accumulate at the point of contact of the active electrode with tissue. However, such configuration may have certain risks, such as muscle stimulation (which may result in muscle contractions); cardiac interference, etc. ; burns or tissue or other thermal tissue damage (e.g., if the grounding pad is not properly applied, and/or from leakage of current outside the targeted area and/or from imprecise control of the electrical current); electrical and/or electromagnetic interference with other medical devices (e.g., pacemakers, defibrillators, monitoring devices, imaging equipment, etc.); or other undesirable and/or adverse effects.

Bipolar devices have several advantages over monopolar devices. As with monopolar devices, the electrical current may be supplied by an electrosurgical energy generator. However, because bipolar devices utilize two electrodes (an active electrode and a return electrode) which are positioned generally in close proximity on (such as at the distal end, distalmost end, tip, etc., of) the device, electrical current flows only between the two electrodes of the bipolar device. The risk of unintended burns or electrical damage to surrounding tissue is thus lower than with monopolar devices in which the current must pass from the active electrode (within the patient) through the patient's tissue and to the grounding pad (outside the patient). The electrical current thus passes across a further distance from the active electrode of the monopolar device to the grounding pad than the distance between the return electrode and the active electrode of a bipolar device. As may be appreciated, the close arrangement of the electrodes of a bipolar device concentrates electrical energy so it does not spread, thereby reducing potential damage and/or interference with other equipment which may occur by the transmission of energy necessary with a monopolar device. Moreover, the close arrangement of the electrodes may allow for finer precision in application of energy from the active electrode to target tissue.

In some aspects, an electrosurgical device formed in accordance with various principles of the present disclosure is a bipolar electrosurgical device utilizing a pair of electrically-conductive components (e.g., functioning as electrodes) to energize the operable end of the electrosurgical device in a bipolar manner. In some aspects, use of a bipolar electrosurgical device is desirable because of the reduced risks presented by a bipolar electrosurgical device compared to a monopolar electrosurgical device.

In some aspects, the pair of electrically conductive components of a bipolar electrosurgical device formed in accordance with various principles of the present disclosure are movable with respect to each other to control the bipolar energy utilizable at the operable end of the device. In some aspects, the pair of electrically-conductive components are movable with respect to each other to shift the operable end of the device into different configuration for performing different operations during a medical procedure. For instance, the electrosurgical device may include a first electrically-conductive component shaped for use as an electrosurgical knife, and a second electrically-conductive component movable with respect to the first electrically-conductive component. In some aspects, movement of the first and second electrically-conductive components with respect to each other allows the electrosurgical device to be used as a grasper. In some aspects, if energy is transmitted through the first and second electrically-conductive components spaced apart from each other and with tissue therebetween, the operable end of the electrosurgical device may be used as a coagulation device with respect to the tissue between the electrically-conductive components. Other configurations and uses of an electrosurgical device formed in accordance with various principles of the present disclosure may be appreciated by those of ordinary skill in the art.

Various embodiments of electrosurgical devices, and associated systems and methods, will now be described with reference to examples illustrated in the accompanying drawings. It should be understood that various features, structures, concepts, and/or characteristics of disclosed embodiments are independent of and separate from one another, and may be used or present individually or in various combinations with one another to create alternative embodiments which are considered part of the present disclosure. Therefore, the present disclosure is not limited to only the embodiments specifically described herein, as it would be too cumbersome to describe all of the numerous possible combinations and subcombinations of features, structures, concepts, and/or characteristics, and the examples of embodiments disclosed herein are not intended as limiting the broader aspects of the present disclosure. It should be appreciated that various dimensions provided herein are examples and one of ordinary skill in the art can readily determine the standard deviations and appropriate ranges of acceptable variations therefrom which are covered by the present disclosure and any claims associated therewith. The following description is of illustrative examples of embodiments only, and is not intended as limiting the broader aspects of the present disclosure.

It will be appreciated that common features in the drawings are identified herein and in the drawings by common reference elements and, for the sake of brevity and convenience, and without intent to limit, the descriptions of the common features are generally not repeated. For purposes of clarity, not all components having the same reference number are numbered if numbered in other figures showing such component. Moreover, similar elements may be indicated by the same reference number, with or without an accompanying letter associated with each similar element.

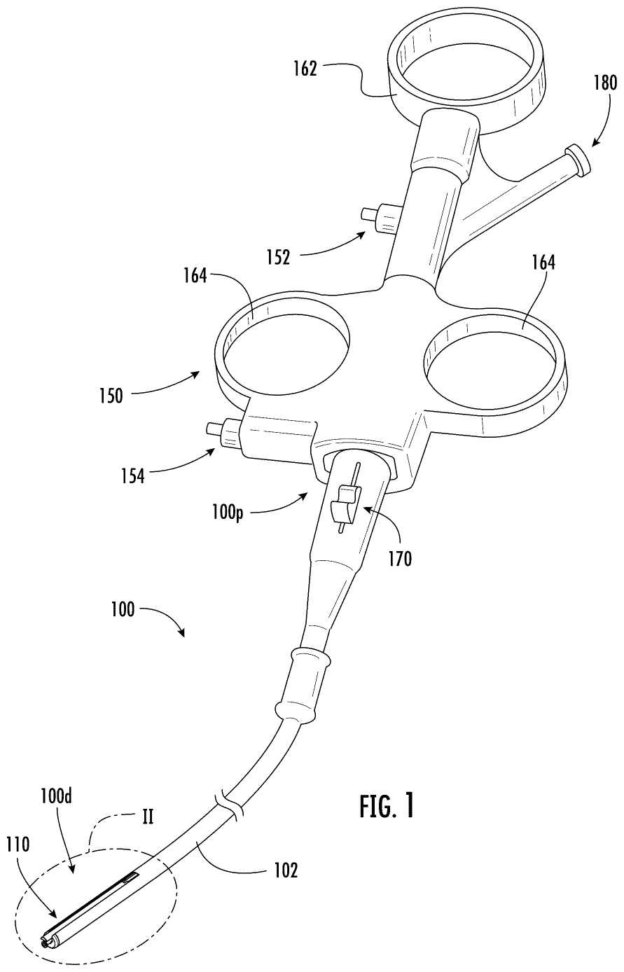

Turning now to the drawings, an example of an embodiment of an electrosurgical device 100 formed in accordance with various principles of the present disclosure is illustrated in FIG. 1. The electrosurgical device 100 includes an elongate member 102 which is sized and sufficiently flexible to be inserted into a natural opening or orifice of a patient's body and navigated into the patient's body (e.g., through curved and/or tortuous passages) to a target site within the patient's body. The elongate member 102 extends to a distal end 100d of the electrosurgical device 100 at which an operable element 110 of the electrosurgical device 100 is provided. In some aspects, a control handle 150 is provided along the proximal end 100p of the electrosurgical device 100. The control handle 150 may be used to control various components and/or features and/or functions of the electrosurgical device 100, as described in further detail below.

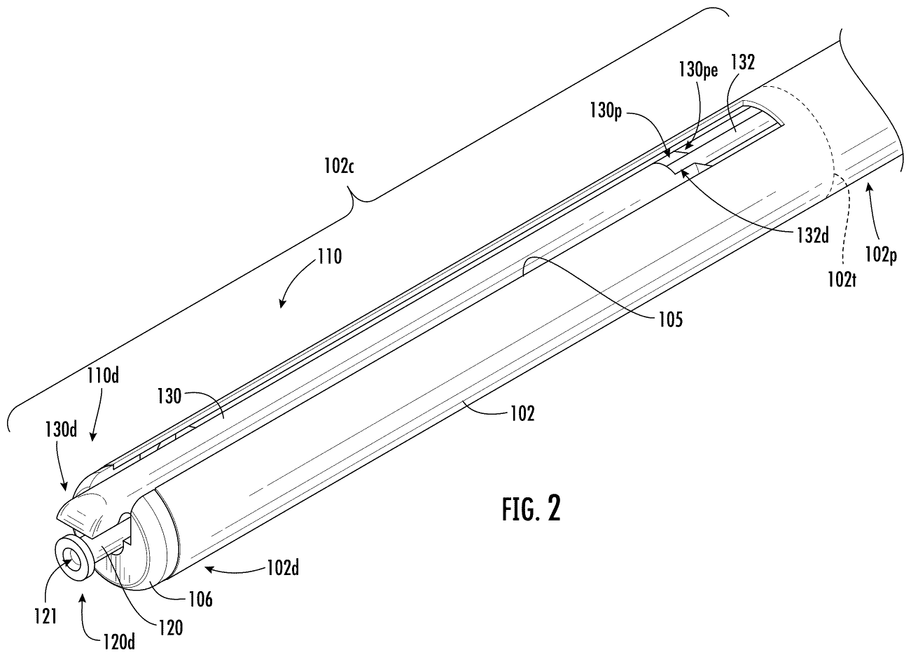

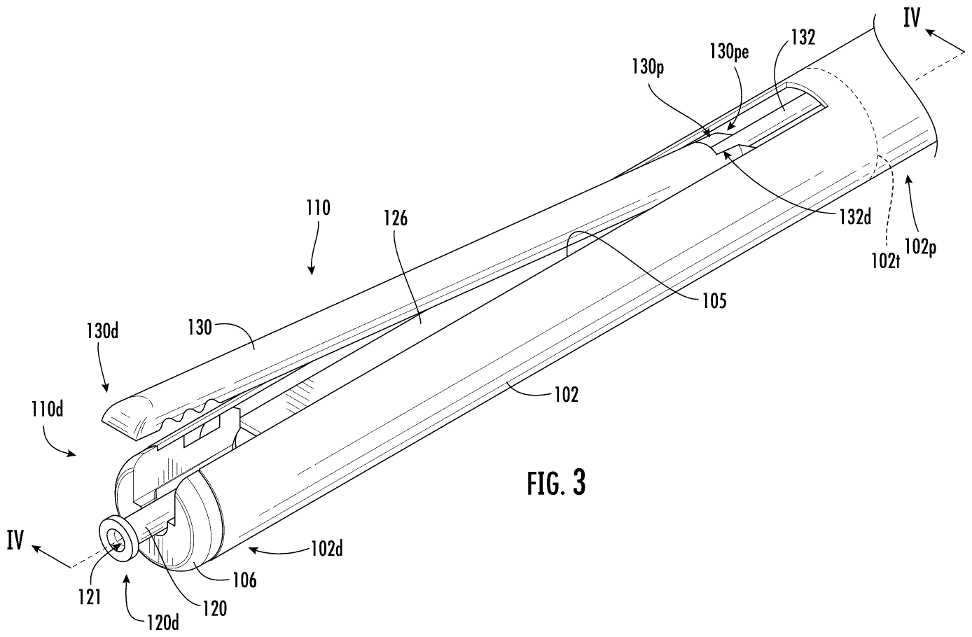

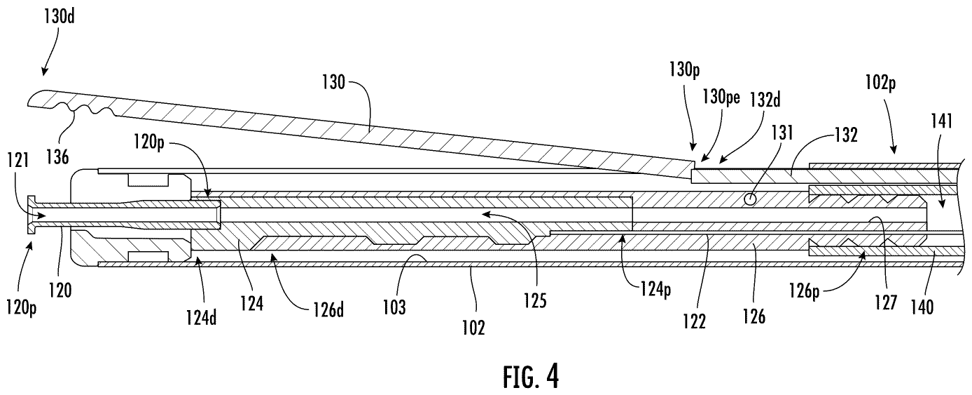

The operable element 110 of the example of an embodiment of an electrosurgical device 100 is illustrated in further detail in FIG. 2, showing the distal end 102d of the elongate member 102 as forming an outer extrusion or housing over the components of the operable element 110. At least the distal end 102d of the elongate member 102, along the operable element 110, is tubular, defining a lumen 103 therethrough in which components of the operable element 110 may be provided (such as may be seen in the cross-sectional view illustrated in FIG. 4, taken along line IV-IV of FIG. 3). In some aspects, a separately formed distal tip 106 may be inserted into the open distal end 102d of the elongate member 102. The distal tip 106 may be rounded or otherwise formed with an atraumatic, blunt end to assist with navigating the elongate member 102 within a patient's body. In some aspects, the elongate member 102 and the distal tip 106 are formed of similar polymers or are otherwise configured to be readily secured together. In some aspects, the elongate member 102 and the distal tip 106 are bonded together, such as with an adhesive/glue or otherwise melded together. In some aspects, the elongate member 102 may be melted, reflowed, or otherwise formed over the distal tip 106. In some aspects, the distal tip 106 is formed of ceramic or another material more rigid and heat resistant than the material of the elongate member 102. In some aspects, ridges, ribs, grooves, dimples, or other surface features are formed in the exterior surface of the distal tip 106 to enhance coupling of the elongate member 102 thereto to resist undesired and/or inadvertent separation of the distal tip 106 from the elongate member 102. In some aspects, the distal tip 106 is mechanically coupled or fixed with respect to the elongate member 102, such as with an interference fit, a press-fit, etc., sufficiently strong to resist undesired and/or inadvertent separation of the distal tip 106 from the elongate member 102. In some aspects, a distal portion 102c of the elongate member 102 is formed of a ceramic or other material resistant to melting when the operable element 110 is in use (e.g., when configured to coagulate tissue when the second electrically-conductive component 130 is moved apart from the first electrically-conductive component 120). In some aspects, the extent of the distal portion 102c is from the distalmost end of the operable element 110 to a proximal-most end 130pe of the second electrically-conductive component 130, or even further proximal thereto, such as to a transition 102t (indicated schematically by a broken line) between the distal end 102d and the proximal end 102p. In some aspects, the length of the distal portion 102c is selected so as not to interfere with the remaining proximal portion of the elongate member 102 (which is typically more flexible than a distal portion 102c formed of ceramic) from being able to be passed through a medical scope in a tortuous anatomy. For instance, the length of the distal portion 102c may be approximately 1-2 inches (2.54-5.08 cm).

The example of an embodiment of an operable element 110 illustrated in FIG. 1, and in greater detail in FIGS. 2-5, includes a first electrically-conductive component 120 and a second electrically-conductive component 130. In order to function as a bipolar electrosurgical device 100, one of the electrically-conductive components is configured and arranged to be an active electrode, and the other of the electrically-conductive components is configured and arranged to be a return electrode. The first electrically-conductive component 120 and the second electrically-conductive component 130 are spaced apart from each other and contacted with tissue, through which current flows (from the first electrically-conductive component 120 to the second electrically-conductive component 130) to deliver energy to the tissue to perform the desired procedure with the operable element 110 of the electrosurgical device 100.

In the example of an embodiment of an operable element 110 illustrated in FIGS. 2-5, the first electrically-conductive component 120 is operably coupled to an electrosurgical energy generator to be configured and arranged to be the active electrode of the bipolar operable element 110, and the second electrically-conductive component 130 is operably coupled to the electrosurgical energy generator to be configured and arranged to be the return electrode of the bipolar operable element 110. The electrosurgical energy generator may be any of a variety of electrosurgical energy generators such as known to those of ordinary skill in the art, the present disclosure not being limited in this regard. The electrosurgical energy generator may be operably coupled with the first electrically-conductive component 120 and the second electrically-conductive component 130 via the control handle 150 in a manner such as known to those of ordinary skill in the art, the present disclosure not being limited in this regard. For instance, in the example of an embodiment illustrated in FIG. 1, an electrical connector 152 is provided on the control handle 150 to electrically couple the electrically-conductive component 120 with an electrosurgical energy generator, and an electrical connector 154 is provided on the control handle 150 to electrically couple the electrically-conductive component 130 with the electrosurgical energy generator. In some aspects, an actuator or controller, such as a foot pedal, is used to control the application or flow of energy from the electrosurgical energy generator to the electrosurgical device 100, such as in a manner known to those of ordinary skill in the art, the present disclosure not being limited in this regard. In some aspects, the amount of energy delivered by the electrosurgical energy generator to the operable element 110 is variable, depending on the operation mode of the operable element 110 (cutting, or coagulation, or otherwise).

In some aspects, the first electrically-conductive component 120 is electrically coupled/connected to the electrosurgical energy generator via one or more electrically conductive elements 122, 124 (illustrated in FIG. 4) through which current may be supplied from the electrosurgical energy generator (e.g., via the electrical connector 152 on the control handle 150) to facilitate the conduction of current to the operable element 110 to energize the first electrically-conductive component 120 to perform the desired procedure therewith. In some aspects, reference to the first electrically-conductive component 120 herein may be interpreted as reference to an assembly of the first electrically-conductive component 120 as well as one or both of the electrically conductive elements 122, 124, as may be appreciated by one of ordinary skill in the art, such as in view of the context of such reference. Typically, an elongated electrically conductive element extends between the active electrode at the distal end of an electrosurgical device and an electrosurgical energy generator. In the example of an embodiment of an electrosurgical device 100 illustrated in FIGS. 1-5, an elongated electrically conductive element 122 is electrically coupled to the first electrically-conductive component 120, such as illustrated in FIG. 4, and extends proximally through the electrosurgical device 100 for proximal connection with an electrosurgical energy generator outside the patient's body (e.g., via an electrical connection provided at or along the control handle 150). In some aspects, the elongated electrically conductive element 122 is in the form of an electrically conductive wire which may be sufficiently flexible to flex with the elongate member 102. In some aspects, the longitudinal extent of the first electrically-conductive component 120 may be limited to the distal end 102d of the elongate member 102. For instance, in some aspects, the first electrically-conductive component 120 may be formed from a very thin tube, such as a hypotube, which might present various assembly and electrical connection challenges. In some aspects, the first electrically-conductive component 120 may be electrically conductively interconnected with (e.g., mounted with respect to) an electrically conductive hub 124, and then the electrically conductive hub 124 is electrically interconnected with the elongated electrically conductive element 122. More particularly, as illustrated in FIG. 4, the proximal end 120p of the first electrically-conductive component 120 may be mounted with respect to a distal end 124d of the electrically conductive hub 124 by being inserted into a lumen 123 defined through the electrically conductive hub 124, and optionally press-fit, interference, fit, welded, etc., therein. The electrically conductive hub 124 may have wider diameter, wall thickness, resistance to deformation, etc.. than the first electrically-conductive component 120 to provide structural support and resistance to deformation to the first electrically-conductive component 120. It will be appreciated that the electrically conductive connections between the first electrically-conductive component 120, the electrically conductive hub 124, and the elongated electrically conductive element 122 may be effected in any manner known to those of ordinary skill in the art, the present disclosure not being limited in this regard. Such electrically interconnected and conductive assembly may serve to stabilize the first electrically-conductive component 120 with respect to the elongate member 102 (and optionally also with respect to the distal tip 106) and/or to facilitate electrical coupling (e.g., welding) of the elongated electrically conductive element 122 with the first electrically-conductive component 120.

In the example of an embodiment of an operable element 110 illustrated in FIGS. 2-5, the second electrically-conductive component 130 is configured and arranged to be the return electrode of the bipolar operable element 110. The close proximity of the first electrically-conductive component 120 and the second electrically-conductive component 130 (in terms of electrical properties, as appreciated by those of ordinary skill in the art) allow energy from the first electrically-conductive component 120 to be passed through tissue positioned adjacent, in contact, and/or between the first electrically-conductive component 120 and the second electrically-conductive component 130, and to be dissipated or absorbed at or by the second electrically-conductive component 130 by completing the electrical circuit as the second electrically-conductive component 130 is electrically connected to the electrosurgical energy generator via an elongate control element 132 (e.g., via the electrical connector 154 along the control handle 150). As such, the bipolar configuration reduces energy transmission through the body of the patient (such as compared with monopolar devices), with accompanying benefits appreciable by those of ordinary skill in the art.

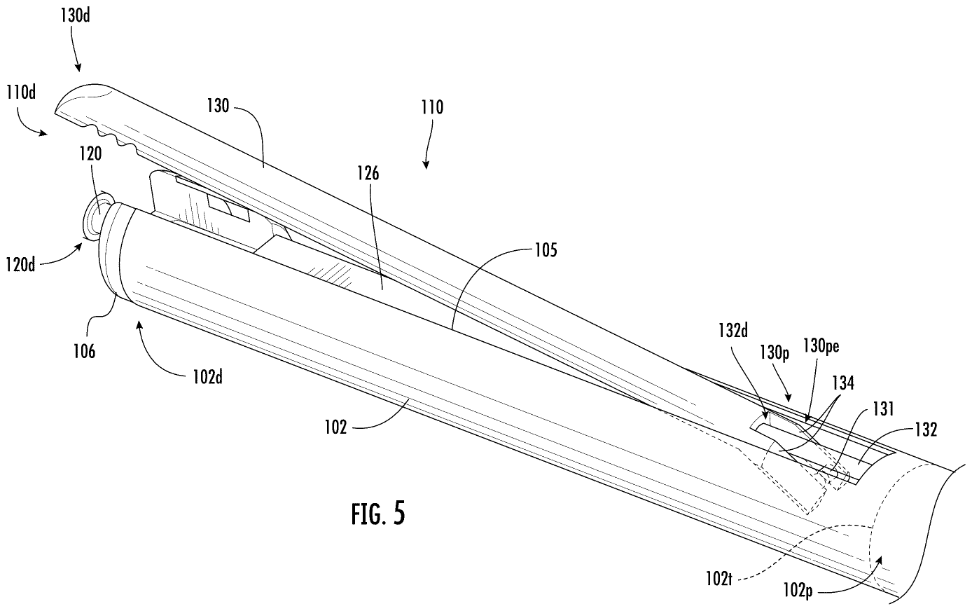

In accordance with various principles of the present disclosure, the first electrically-conductive component 120 and the second electrically-conductive component 130 may be movable with respect to each other, such as may be appreciated with reference to FIG. 2 as compared to FIG. 3 and FIG. 5. More particularly, in the example of an embodiment illustrated in FIGS. 2-5, the second electrically-conductive component 130 is movable with respect to the first electrically-conductive component 120. Even more particularly, the proximal end 130p of the second electrically-conductive component 130 of the illustrated example of an embodiment is pivotably coupled with respect to the first electrically-conductive component 120. In the example of an embodiment illustrated in FIG. 4 and FIG. 5, the proximal end 130p of the second electrically-conductive component 130 may be pivotably coupled with respect to the first electrically-conductive component 120, and/or the elongate member 102, about a pivot pin 131. In some aspects, the elongate control element 132 is used to move the second electrically-conductive component 130 with respect to the first electrically-conductive component 120, as described in further detail below. The movement of the first electrically-conductive component 120 with respect to the second electrically-conductive component 130 may determine the operation and/or use and/or function of the operable element 110, as described in further detail below.

In some aspects, an overmold 126 is provided or formed with respect to the first electrically-conductive component 120 and the second electrically-conductive component 130 to facilitate movable (e.g., pivotable) coupling of the second electrically-conductive component 130 with the first electrically-conductive component 120. For instance, as illustrated in FIG. 4, an overmold 126 may be provided or formed over the electrically conductive hub 124 to which the first electrically-conductive component 120 is electrically conductively coupled. The pivot pin 131 provided or formed along the proximal end 130p of the second electrically-conductive component 130 may be embedded in the overmold 126 in a manner allowing rotational movement within the overmold 126 to allow pivoting of the second electrically-conductive component 130. In the example of an embodiment of an operable element 110 illustrated in FIG. 5, the proximal end 130p of the second electrically-conductive component 130 may be in the form of or include a clevis with a pair of legs 134 secured to opposite ends of the pivot pin 131 and positioned outside the overmold 126 to operably couple the pivot pin 131 with the remaining distally extending portion of the second electrically-conductive component 130 (extending distally from the pivot pin 131).

In some aspects, a slot or cut-out 105 is defined along the distal end 102d of the elongate member 102, such as to facilitate stowing of the second electrically-conductive component 130 therein, such as to form a compact configuration such as illustrated in FIG. 2. The “compact” configuration may be appreciated as relative to an operating configurations such as illustrated in FIGS. 3-5. In some aspects, the above-described overmold 126 is thermally insulating, such as formed of a thermally insulative plastic or ceramic material. In some aspects, the above-described overmold 126 may be formed of a nonconductive/insulative material, at least in the region of the cut-out 105, to electrically isolate portions of the first electrically-conductive component 120 from the second electrically-conductive component 130, such as in the vicinity of the cut-out 105, as may be appreciated with reference to FIG. 4. As may be appreciated with reference to FIG. 3 and FIG. 5, if electrical conductivity between the first electrically-conductive component 120 and the second electrically-conductive component 130 is not desired along the full length of the cut-out 105 (e.g., along the electrically conductive hub 124), the overmold 126 is provided along the cut-out 105 to inhibit or prevent transmission of energy from the first electrically-conductive component 120 to the second electrically-conductive component 130 along the cut-out 105. However, it will be appreciated that the distance of the distal end 126d of the overmold 126 from the distal end 124d of the electrically conductive hub 124 and/or the distal end 120d of the first electrically-conductive component 120 may be selected to alter the longitudinal extent of electrical conductivity between the first electrically-conductive component 120 and the second electrically-conductive component 130. The distal end 126d of the overmold 126 may thus extend up to (substantially at the same longitudinal extent) or proximal to (spaced proximally from) the distal end 124d of the electrically conductive hub 124.

In the example of an embodiment of an operable element 110 illustrated in FIGS. 2-5, at least one elongate control element 132 is operably coupled with the proximal end 130p of the second electrically-conductive component 130 such that an operational force (e.g., a proximal pulling force) applied to the elongate control element 132 from outside the patient (e.g., by a medical professional) may be transmitted to the second electrically-conductive component 130 to control movement of the second electrically-conductive component 130. For instance, the elongate control element 132 may be in the form of a pull wire with a distal end 132d operably associated with (e.g., fixedly coupled to) the proximal end 130p of the second electrically-conductive component 130 (such as by an adhesive, welding, brazing, etc.). In some aspects, the proximal end 130p of the second electrically-conductive component 130 is angled and/or arched, such as illustrated in FIG. 5, to create a moment arm between the pivot pin 131 and the proximal end 130p of the second electrically-conductive component 130 operably coupled with the distal end 132d of the elongate control element 132 to effect movement (e.g., pivoting) of the second electrically-conductive component 130, such as upon pulling the elongate control member 132 proximally. It will be appreciated that the distal end 132d of the elongate control element 132 should be securely attached to the proximal end 130p of the second electrically-conductive component 130 to transmit proximal forces thereto without detaching from the second electrically-conductive component 130. Such attachment may be achieved by adhesive, mechanical interfitting (e.g., shapes which interlock; the distal end 132d of the elongate control element 132 being looped in and around an aperture in the proximal end 130p of the second electrically-conductive component 130 and/or around a distal side of an enlarged element formed on or along the proximal end 130p of the second electrically-conductive component 130; and/or other modes of mechanically interconnecting elements known to those of ordinary skill in the art), welding, brazing, or any other manner known to those of ordinary skill in the art, the present disclosure not being limited in this regard. It will further be appreciated that the elongate control element 132 should be made of a material with sufficient tensile as well as column strength to be able to transmit pulling or pushing forces longitudinally along the electrosurgical device 100 to actuate movement of the second electrically-conductive component 130. In some aspects, the elongate control element 132 is formed from an electrically conductive wire similar to the electrically conductive wire from which the elongated electrically conductive element 122 is formed. In some aspects, the diameter of the elongate control element 132 is larger than the diameter of the elongated electrically conductive element 122. For example, the outer diameter of the operable element 110 of the electrosurgical device 100 is targeted at under approximately 2.6 mm, and the elongated electrically conductive element 122 may have a diameter of up to approximately 0.254 mm (and generally larger than approximately 0.254 mm, and typically approximately 0.0762 mm), whereas the outer diameter of the elongate control element 132 is preferably larger than approximately 0.254 mm, and even up to approximately 0.762 mm.

In some aspects, movement of the second electrically-conductive component 130 may be controlled at a proximal end 100p of the electrosurgical device 100, such as along the control handle 150 (illustrated in FIG. 1) with one or more actuators along the control handle 150. In the example of an embodiment of a control handle 150 illustrated in FIG. 1, an operable element actuator 160 includes a thumb ring 162 and one or more finger loops 164 may be arranged and configured and operably associated with the second electrically-conductive component 130 to control, remotely from outside the patient, movement of the second electrically-conductive component 130 with respect to the first electrically-conductive component 120 while the operable element 110 is within the patient. For instance, the elongate control element 132 may be operably coupled with the finger loops 164 so that proximal movement of the finger loops 164 (e.g., towards the thumb ring 162) causes movement of the second electrically-conductive component 130 (e.g., away from the first electrically-conductive component 120). As may be appreciated, reference to a “thumb” ring 162 and reference to “finger” loops 164 are for the sake of convenience and are not intended to restrict operation or use thereof to engagement with a thumb or fingers.

In accordance with various principles of the present disclosure, the second electrically-conductive component 130 may be movable between a first configuration, such as illustrated in FIG. 2, and a second configuration, such as illustrated in FIGS. 3-5. It will be appreciated that the specific distances between the first electrically-conductive component 120 and the second electrically-conductive component 130 in the different configurations may vary from those illustrated in the accompanying figures, the present disclosure not being limited by specific distances. Movability of the first electrically-conductive component 120 and the second electrically-conductive component 130 with respect to each other allows for different configurations of the operable element 110 which may allow different uses or functionalities or modes of operation of the operable element 110. As noted above, optionally, the amount of energy supplied by the electrosurgical energy generator to the operable element 110 may be varied depending on the desired use of the operable element 110, such as may be determined based on the configuration of the operable element 110, as described below. In some aspects, in the first configuration, the first electrically-conductive component 120 and the second electrically-conductive component 130 are positioned with respect to each other to configure the operable element 110 in a first configuration operable to be perform a first type of action during a medical procedure. In some aspects, in the second configuration, the first electrically-conductive component 120 and the second electrically-conductive component 130 are positioned with respect to each other to configure the operable element 110 in a second configuration different from the first configuration and operable to be perform a second type of action, which is different from the first type of action, during a medical procedure.

In some aspects, in the first configuration of the operable element 110, such as illustrated in FIG. 2, the distal end 130d of the second electrically-conductive component 130 is adjacent yet sufficiently spaced apart from the distal end 120d of the first electrically-conductive component 120. For instance, the distal end 130d of the second electrically-conductive component 130 may be spaced apart from the distal end 120d of the first electrically-conductive component 120 by a small gap, so as not to not create a short circuit, and across which current cannot typically flow without a conductor extending thereacross (a distance which readily determinable by one of ordinary skill in the art). In some aspects, the distal end 130d of the second electrically-conductive component 130 may be spaced apart from the distal end 120d of the first electrically-conductive component 120 by at least about 0.05″ (1.27 mm) to about 0.06″ (1.524 mm). When tissue (which is generally electrically conductive) is contacted by the distal ends 120d, 130d of the first electrically-conductive component 120 and the second electrically-conductive component 130, the circuit between the first electrically-conductive component 120 and the second electrically-conductive component 130 is closed so that energy may pass from the first electrically-conductive component 120 to the second electrically-conductive component 130.

In some aspects, in the first configuration, the distal end 110d of the operable element 110 may be used as an electrosurgical cutting device. For instance, the distal end 110d of the operable element 110, and thus the distal end 120d of the first electrically-conductive component 120 and the distal end 130d of the second electrically-conductive component 130, may be contacted with tissue. Energy is delivered to the operable element 110, such as upon actuation of an electrosurgical energy generator, such as by operation of a foot pedal or other actuator operably associated with the electrosurgical energy generator, at an appropriate level of energy. The energy (e.g., current) is supplied to the first electrically-conductive component 120 and passed through the contacted tissue, which extends across a gap between the first electrically-conductive component 120 and the second electrically-conductive component 130, to the second electrically-conductive component 130. The flow of energy through the tissue closes the circuit of the operable element 110 of the bipolar electrosurgical device 100 to effect cutting of the tissue. For instance, the resistance of the tissue may cause the electrical current to generate resistive heat in the tissue resulting in separation or cutting of the contacted tissue. In some aspects, the electrosurgical device 100 may be considered an electrosurgical cutting device with a movable arm operably associated with the cutting device, the movable arm laterally movable with respect to the cutting device.

In some aspects, the first electrically-conductive component 120 of the example of an embodiment of an operable element 110 illustrated in FIGS. 1-5 is configured as a tissue-cutting element. For instance, the distal end 120d of the first electrically-conductive component 120 may be formed into a cutting edge configuration, such as with a T shape longitudinal cross-section (with optionally a circular cross-section orthogonal to the longitudinal cross-section), as may be appreciated with reference to FIG. 4, or a tapered or other shape conducive to use as a cutting element, such as known to those of ordinary skill in the art. In some aspects, the distal end 130d of the second electrically-conductive component 130 is tapered or rounded or otherwise sized, shaped, configured, positioned, and/or dimensioned to form a distal end 110d of the operable element 110 optimized for cutting tissue, such as may be appreciated by those of ordinary skill in the art. In some aspects, the first electrically-conductive component 120 is longitudinally translatable (e.g., 1-3 mm) with respect to (e.g., within) the elongate member 102 to vary the distance of the distal end 120d of the first electrically-conductive component 120 from the distal tip 106 of the elongate member 102 (such as to adjust the length of the cutting surface/edge defined along the distal end 120d of the first electrically-conductive component 120). In some aspects, an actuator 170 is provided on the control handle 150 and operably associated with the first electrically-conductive component 120 such that movement of the actuator 170 controls movement of the first electrically-conductive component 120 relative to the elongate member 102. In some aspects, the actuator 170 is a slider which is longitudinally slidable along the control handle 150 to effect longitudinal translation of the first electrically-conductive component 120 with respect to the elongate member 102. In some aspects, the actuator 170 is operably coupled with the elongate member 102, so that movement of the actuator 170 moves the elongate member 102 relative to the first electrically-conductive component 120. For instance, longitudinal movement of the elongate member 102 relative to the first electrically-conductive component 120 allows the first electrically-conductive component 120 (and, optionally, the second electrically-conductive component 130 as well) to be within or outside the elongate member 102. In some aspects, the first electrically-conductive component 120 and the second electrically-conductive component 130 are extendable out of the elongate member 102 in unison to allow user control of operation of the second electrically-conductive component 130 with respect to the first electrically-conductive component 120.

In some aspects, the first electrically-conductive component 120 defines a lumen 121 therethrough, through which a fluid (e.g., air, saline, etc.) may be delivered. The fluid may be delivered from a fluid source (any known to those of ordinary skill in the art, the present disclosure not being limited in this regard) to flush a target site, to be used as a lifting agent (e.g., to inject in tissue to create a raised area or “bleb” to facilitate cutting of the tissue), etc. In some aspects, an inner extrusion 140, defining a lumen 141 therethrough, extends proximally from the first electrically-conductive component 120 and longitudinally through the elongate member 102 to a port 180 along the control handle 150. The port 180 may be fluidly coupled with fluid source so that fluid may be delivered via the port 180 through the lumen 141 of the inner extrusion 140, to and through the lumen 121 through the first electrically-conductive component 120, and out the distal end 120d of the first electrically-conductive component 120. In some aspects, the proximal end 126p of the overmold 126 is secured with respect to the inner extrusion 140, such as by being inserted into and secured with respect to the lumen 141 of the inner extrusion 140 (e.g., sufficiently so that the inner extrusion 140 and the overmold 126 do not separate). In some aspects, the distal end 140d of the inner extrusion 140 and the proximal end 126p of the overmold 126 are secured together by an interference fit or friction fit, or adhered (e.g., with glue, adhesive, etc.) or otherwise bonded (e.g., a material or chemical bond such as achieved by melting and reflowing one or both elements, such as if made from similar materials). In some aspects, the lumen 141 of the inner extrusion 140 is fluidly communicated with the lumen 121 through the first electrically-conductive component 120 via a lumen 127 defined through the overmold 126 and a lumen 125 defined through the electrically conductive hub 124. In some aspects, the inner extrusion 140 is in the form of an elongate member which is sufficiently flexible to be inserted into and navigated within a patient's body (e.g., through curved or tortuous passages within the patient's body).

In some aspects, the second configuration of the operable element 110, with the second electrically-conductive component 130 in a second configuration with respect to the first electrically-conductive component 120, allows a different mode of operation of the operable element 110. In the example of an embodiment illustrated in FIGS. 3-5, the first electrically-conductive component 120 and the second electrically-conductive component 130 may be sufficiently spaced apart when the second electrically-conductive component 130 is in a second configuration to allow tissue to be positioned between the first electrically-conductive component 120 and the second electrically-conductive component 130. In such configuration, the operable element 110 is operable as an electrocoagulation device. Electrical current (e.g., supplied by an electrosurgical energy generator, such as described above) flows from the first electrically-conductive component 120 (e.g., from the electrosurgical energy generator via the electrical connector 152), through the tissue positioned between the first electrically-conductive component 120 and the second electrically-conductive component 130, and to the second electrically-conductive component 130 (and, e.g., returned, such as via the electrical connector 154, to the electrosurgical energy generator). In some aspects, the energy level may be selected (e.g., by a controller positioned along the control handle 150 of the electrosurgical device 100, or along the electrosurgical energy generator or controller thereof, such as a foot pedal, or otherwise) to achieve the desired operative effect on the tissue (e.g., coagulation or otherwise). In some aspects, the surface 136 of the second electrically-conductive component 130 which contacts tissue may be crenulated, wavy, toothed or otherwise textured to enhance grasping of tissue and/or transmission of energy for achieving the desired effect with respect to the tissue positioned between the first electrically-conductive component 120 and the second electrically-conductive component 130.

In some aspects, energy need not be supplied to the first electrically-conductive component 120 and the second electrically-conductive component 130. In such instance, the operable element 110 may be used in yet another mode, such as by using first electrically-conductive component 120 and the second electrically-conductive component 130 simply to push tissue or move vessels or other matter, such as when in the first configuration. In some aspects, the first electrically-conductive component 120 and the second electrically-conductive component 130 may be used (particularly if adjacent, and optionally contacting, each other) as a blunt tissue dissector. In yet another aspect of the present disclosure, the first electrically-conductive component 120 and the second electrically-conductive component 130 may be operable in yet another mode, such as a grasper. For instance, the first electrically-conductive component 120 and the second electrically-conductive component 130 may be movable with respect to each other to receive and then grasp tissue, vessels (e.g., blood vessels), or other biological or anatomical tissue, and/or medical devices, therebetween, and optionally also to move the grasped element. Such operation of the first electrically-conductive component 120 and the second electrically-conductive component 130 may be without transmission of energy to the first electrically-conductive component 120 and the second electrically-conductive component 130.

It should be appreciated that medical devices and systems described herein may be used in a variety of medical procedures performed in connection with any of a variety of anatomical structures or systems, including, for example, the gastrointestinal system, the abdominal cavity, digestive system, urinary tract, reproductive tract, respiratory system, cardiovascular system, circulatory system. It should be appreciated that medical devices and systems described herein may be used in conjunction with any of a variety of medical devices for navigating body lumens, including, for example, catheters, endoscopes, ureteroscopes, bronchoscopes, colonoscopes, arthroscopes, cystoscopes, hysteroscopes, etc. The disclosed medical devices and systems may also be inserted via different access points and approaches, e.g., percutaneously, endoscopically, laparoscopically, or combinations thereof.

In view of the above, it will be appreciated that various principles of the present disclosure may be achieved with various components, elements, arrangements, configurations, etc., other than those described above. For instance, different configurations of control handles and associated actuators or control elements therealong, different formations and/or operable couplings of electrically-conductive components, and other variations to the above described elements of an electrosurgical device may be substituted for the above described elements, etc., without departing from the principles of the present disclosure described herein. Thus, it will be appreciated that all apparatuses and methods discussed herein are examples of apparatuses and/or methods implemented in accordance with one or more principles of this disclosure. These examples are not the only way to implement these principles but are merely examples, not intended as limiting the broader aspects of the present disclosure. Thus, references to elements or structures or features in the drawings must be appreciated as references to examples of embodiments of the disclosure, and should not be understood as limiting the disclosure to the specific elements, structures, or features illustrated. Other examples of manners of implementing the disclosed principles will occur to a person of ordinary skill in the art upon reading this disclosure. It will further be appreciated that the various features described herein may be used singly or in any combination thereof. Therefore, the present invention is not limited to only the embodiments specifically described herein. It is therefore to be understood by one of ordinary skill in the art that the present discussion is a description of illustrative examples of embodiments only, and is not intended as limiting the broader aspects of the present disclosure. Various further benefits of the various aspects, features, components, and structures of electrosurgical devices, systems, and methods such as described above, in addition to those discussed above, may be appreciated by those of ordinary skill in the art.

The foregoing discussion has broad application and has been presented for purposes of illustration and description and is not intended to limit the disclosure to the form or forms disclosed herein. It will be understood that various additions, modifications, and substitutions may be made to embodiments disclosed herein without departing from the concept, spirit, and scope of the present disclosure. In particular, it will be clear to those skilled in the art that principles of the present disclosure may be embodied in other forms, structures, arrangements, proportions, and with other elements, materials, and components, without departing from the concept, spirit, or scope, or characteristics thereof. For example, various features of the disclosure are grouped together in one or more aspects, embodiments, or configurations for the purpose of streamlining the disclosure. However, it should be understood that various features of the certain aspects, embodiments, or configurations of the disclosure may be combined in alternate aspects, embodiments, or configurations. While the disclosure is presented in terms of embodiments, it should be appreciated that the various separate features of the present subject matter need not all be present in order to achieve at least some of the desired characteristics and/or benefits of the present subject matter or such individual features. One skilled in the art will appreciate that the disclosure may be used with many modifications or modifications of structure, arrangement, proportions, materials, components, and otherwise, used in the practice of the disclosure, which are particularly adapted to specific environments and operative requirements without departing from the principles or spirit or scope of the present disclosure. For example, elements shown as integrally formed may be constructed of multiple parts or elements shown as multiple parts may be integrally formed, the operation of elements may be reversed or otherwise varied, the size or dimensions of the elements may be varied. Similarly, while operations or actions or procedures are described in a particular order, this should not be understood as requiring such particular order, or that all operations or actions or procedures are to be performed, to achieve desirable results. Additionally, other implementations are within the scope of the following claims. In some cases, the actions recited in the claims can be performed in a different order and still achieve desirable results. The presently disclosed embodiments are therefore to be considered in all respects as illustrative and not restrictive, the scope of the claimed subject matter being indicated by the appended claims, and not limited to the foregoing description or particular embodiments or arrangements described or illustrated herein. In view of the foregoing, individual features of any embodiment may be used and can be claimed separately or in combination with features of that embodiment or any other embodiment, the scope of the subject matter being indicated by the appended claims, and not limited to the foregoing description.

In the foregoing description and the following claims, the following will be appreciated. The phrases “at least one”, “one or more”, and “and/or”, as used herein, are open-ended expressions that are both conjunctive and disjunctive in operation. The terms “a”, “an”, “the”, “first”, “second”, etc., do not preclude a plurality. For example, the term “a” or “an” entity, as used herein, refers to one or more of that entity. As such, the terms “a” (or “an”), “one or more” and “at least one” can be used interchangeably herein. As used in this specification and the appended claims, the term “or” is generally employed in its sense including “and/or” unless the content clearly dictates otherwise. As used herein, the conjunction “and” includes each of the structures, components, features, or the like, which are so conjoined, unless the context clearly indicates otherwise, and the conjunction “or” includes one or the others of the structures, components, features, or the like, which are so conjoined, singly and in any combination and number, unless the context clearly indicates otherwise. All directional references (e.g., proximal, distal, upper, lower, upward, downward, left, right, lateral, longitudinal, front, back, top, bottom, above, below, vertical, horizontal, radial, axial, clockwise, counterclockwise, and/or the like) are only used for identification purposes to aid the reader's understanding of the present disclosure, and/or serve to distinguish regions of the associated elements from one another, and do not limit the associated element, particularly as to the position, orientation, or use of this disclosure. Connection references (e.g., attached, coupled, connected, engaged, joined, etc.) are to be construed broadly and may include intermediate members between a collection of elements and relative movement between elements unless otherwise indicated. As such, connection references do not necessarily infer that two elements are directly connected and in fixed relation to each other. Identification references (e.g., primary, secondary, first, second, third, fourth, etc.) are not intended to connote importance or priority, but are used to distinguish one feature from another.

The following claims are hereby incorporated into this Detailed Description by this reference, with each claim standing on its own as a separate embodiment of the present disclosure. In the claims, the terms “comprises”, “comprising”, “includes”, and “including” do not exclude the presence of other elements, components, features, groups, regions, integers, steps, operations, etc. Additionally, although individual features may be included in different claims, these may possibly advantageously be combined, and the inclusion in different claims does not imply that a combination of features is not feasible and/or advantageous. In addition, singular references do not exclude a plurality. Reference signs in the claims are provided merely as a clarifying example and shall not be construed as limiting the scope of the claims in any way.

What is claimed is:

1. A bipolar electrosurgical device comprising:
an elongate member sufficiently flexible to be deliverable transluminally within a patient to a treatment site;
a first electrically-conductive component; and
a second electrically-conductive component;
wherein said first electrically-conductive component and said second electrically-conductive component are movable with respect to each other between at least a first configuration and a second configuration.

2. The bipolar electrosurgical device of claim 1, wherein said second electrically-conductive component is pivotable with respect to said first electrically-conductive component.

3. The bipolar electrosurgical device of claim 1, wherein the level of energy supplied to said bipolar electrosurgical device is selectable by a medical professional based on the configuration of said electrically-conductive components.

4. The bipolar electrosurgical device of claim 1, wherein in the first configuration of said electrically-conductive components, said electrically-conductive components are usable as an electrosurgical cutting device.

5. The bipolar electrosurgical device of claim 4, wherein in the second configuration of said electrically-conductive components, said electrically-conductive components are usable as an electrocoagulation device with respect to tissue positioned between said first and second electrically-conductive components.

6. The bipolar electrosurgical device of claim 1, wherein in the second configuration of said electrically-conductive components, said electrically-conductive components are usable as an electrocoagulation device with respect to tissue positioned between said first and second electrically-conductive components.

7. The bipolar electrosurgical device of claim 6, wherein said second electrically-conductive component has a surface configured to facilitate grasping of tissue between said second electrically-conductive component and said first electrically-conductive component.

8. The bipolar electrosurgical device of claim 1, wherein said first electrically-conductive component is configured as a cutting device.

9. The bipolar electrosurgical device of claim 8, wherein said first electrically-conductive component defines a lumen therethrough for delivery of a fluid therethrough and out the distal end of the bipolar electrosurgical device.

10. The bipolar electrosurgical device of claim 1, further comprising an elongate control element operably coupled with a proximal end of said second electrically-conductive component and extendable to a proximal end of said bipolar electrosurgical device for application of a force thereto to move said second electrically-conductive component with respect to said first electrically-conductive component.

11. The bipolar electrosurgical device of claim 1, further comprising an insulative component between a portion of said first electrically-conductive component and a portion of said second electrically-conductive component.

12. The bipolar electrosurgical device of claim 11, wherein said first electrically-conductive component is electrically coupled with an electrically conductive hub, and said insulative component is overmolded over said electrically conductive hub and over a portion of a pivot pin operably coupled with a proximal end of said second electrically-conductive component to pivotably couple said second electrically-conductive component with respect to said first electrically-conductive component.

13. A bipolar electrosurgical cutting device comprising:

14.
a first electrode configured to cut tissue; and

15.
a second electrode;

16.
wherein said first electrode and said second electrode are movable with respect to each other.

17. The bipolar electrosurgical cutting device of claim 13, wherein said second electrode is pivotable with respect to said first electrode.

18. The bipolar electrosurgical cutting device of claim 13, wherein said first electrode and said second electrode are movable to a first configuration in which said first electrode and said second electrode are operable to cut tissue with energy supplied to said first electrode and passing through tissue, contacted by said first electrode and said second electrode, to said second electrode.

19. The bipolar electrosurgical cutting device of claim 13, wherein said first electrode and said second electrode are movable to a second configuration in which said first electrode and said second electrode are operable to coagulate tissue positioned therebetween with energy supplied to said first electrode and passing through the tissue and to said second electrode.

20. A method of performing a procedure with respect to tissue within a patient, said method comprising:

21.
delivering an operable element of an electrosurgical device transluminally into a patient and to a treatment site, the operable element comprising a first electrode and a second electrode movable with respect to each other;

22.
providing energy to the first electrode and the second electrode when the operable element is in a first configuration to operate in a first mode with respect to tissue at the treatment site; and

23.
providing energy to the first electrode and the second electrode when the operable element is in a second configuration to operate in a second mode with respect to tissue at the treatment site.

24. The method of claim 17, further comprising providing a first level of energy to the operable element when in the first configuration, and a second level of energy, different from the first level of energy, to the operable element when in the second configuration.

25. The method of claim 17, further comprising cutting tissue in the first mode of operation.

26. The method of claim 17, further comprising coagulating tissue in the second mode of operation.
